(19) United States
(12) Patent Application Publication
(10) Pub. No.: US 2026/0263802 A1
(43) Pub. Date: Sep. 10, 2026

(54) PATIENT-SPECIFIC SEIZURE DETECTION USING VAGAL ELECTRONEUROGRAMS

(71) Applicant: IVEACARE, INC., Roseville, MN (US)

(72) Inventors: Randolph Armstrong, Katy, TX (US); Todd Alan Kerkow, Stacy, MN (US)

(21) Appl. No.: 19/160,972

(22) PCT Filed: Mar. 5, 2024
(86) PCT No.: PCT/US2024/018470

§ 371 Date: Aug. 29, 2025

Related U.S. Application Data
(60) Provisional application No. 63/488,505, filed on Mar. 5, 2023.

(51) Int. Cl. A61N 1/36; A61B 5/00; A61B 5/0205; A61B 5/024; A61B 5/08; A61B 5/294; A61B 5/388; A61N 1/02

(52) U.S. Cl. CPC A61N 1/36064; A61B 5/0205; A61B 5/388; A61B 5/7267; A61N 1/025; A61N 1/36053; A61N 1/36139; A61B 5/0077; A61B 5/02405; A61B 5/08; A61B 5/294

(57) ABSTRACT

A seizure management system can include a first sensor configured to receive electrical signal information from a vagus nerve of a patient and a processor circuit. In an example, the processor circuit can be used to identify, in a training portion of the electrical signal information from the vagus nerve, one or more pre-seizure or intra-seizure signal patterns, and monitor a monitoring portion of the electrical signal information from the vagus nerve for signal characteristics that correspond to the identified pre¬seizure or intra-seizure signal patterns. Responsive to recognizing, in the monitoring portion of the electrical signal information, signal characteristics that correspond to the identified pre-seizure or intra-seizure signal patterns, a vagal nerve stimulation (VNS) therapy can be provided to the patient.

### CLAIM OF PRIORITY

This application is related to and claims priority to U.S. Provisional Application No. 63/488,505, filed on Mar. 5, 2023, and entitled “Patient-specific Seizure Detection using Vagal Electroneurograms,” the entirety of which is incorporated herein by reference.

### FIELD OF THE DISCLOSURE

This document pertains generally, but not by way of limitation, to sensing of neural activity at or near a vagal region, such as for use in providing vagal neural stimulation in a closed-loop manner.

### BACKGROUND

Neurological disorders include conditions that affect the nervous system, such as including the brain, spinal cord, and peripheral nerves. These disorders can manifest through various symptoms, such as cognitive impairments, motor function issues, sensory disruptions, and autonomic nervous system irregularities. The causes of neurological disorders are diverse and can include genetic factors, environmental influences, traumatic injuries, and chronic diseases.

Treatment for neurological disorders varies depending on the specific condition and its severity. Some approaches include pharmacological interventions, physical therapy, and surgical procedures. However, not all patients respond to these treatments, and some may experience side effects or limited improvement in their symptoms.

Epilepsy is a disorder in which nerve cell activity in the brain is disturbed, causing seizures. During a seizure, a person can experience abnormal behavior, symptoms, and sensations, sometimes including loss of consciousness. Epilepsy can be treated by medications and in some cases by surgery, devices, or dietary changes. Though some seizures can be controlled with medication, if medication becomes ineffective, other forms of treatment may be considered, including neurostimulation therapy.

### BRIEF DESCRIPTION OF THE DRAWINGS

To easily identify the discussion of any particular element or act, the most significant digit or digits in a reference number refer to the figure number in which that element is first introduced. The drawings are not drawn to scale.

FIG. 1 illustrates generally an example of a neurostimulation system.

FIG. 2 illustrates generally an example of a tripolar lead assembly.

FIG. 3 illustrates generally example charts showing various physiologic status-indicating signals for a patient.

FIG. 4 illustrates generally an example of a first method that can include using a pattern detection routine to analyze physiologic status information about a patient and control a therapy for the patient.

FIG. 5 illustrates generally an example of a second method that can include identifying a seizure detection pattern.

FIG. 6 illustrates generally an example of a machine in the form of a computer system within which a set of instructions may be executed for causing the machine to perform any one or more of the methodologies discussed herein, according to an example embodiment.

### DETAILED DESCRIPTION

A vagus nerve stimulation (VNS) system can include an implantable pulse generator (IPG), a lead that attaches to the vagus nerve and the IPG, and a programmer used to program or assess the status of IPG. The implantable device, or a treatment system that comprises the device, can be configured to detect physiologic changes as indicators of possible adverse conditions or events. For example, the system can be configured to detect seizure events, progression of depression or other behavioral or mood disorders, progression of rehabilitation such as after a stroke, or to detect other disorders or physiologic effects that may be influenced by vagal nerve activity. For example, information about heart rate increase can be used as a surrogate for a possible seizure event. In response to detecting a specific heart rate increase, the device can provide VNS therapy to the patient. However, such an approach can present various challenges. For example, use of heart rate as a detector may present a high false-positive rate because seizure occurrence may be only loosely correlated with an increase in heart rate.

In another approach, seizure detection can be performed using information about autonomic auras. An autonomic aura can include a manifestation of an epileptic seizure, or precursor to a seizure, pertaining to autonomic nervous system function. In an example, the aura can include effects that are cardiorespiratory (e.g., heart rate variability, palpitations and shortness of breath), gastrointestinal, genitourinary (e.g., genital sensations, urinary urge), or cutaneous (e.g., feeling of warmth or cold), among others. Abdominal auras can include sensations of nausea, pain, or indescribable discomfort in the abdominal or periumbilical area that can be static, rise to the chest and throat, or descend into the lower abdominal region.

The present inventors have recognized that a problem to be solved includes improving seizure detection accuracy. The problem can further include providing an effective seizure therapy in response to detected seizure events, such as without unnecessarily increasing a magnitude or duration of VNS therapy. The present inventors have recognized that a solution to these and other problems can include or use a system that includes an implantable neurostimulation device and one or more sensors, such as can be used together to provide closed-loop therapy for seizure intervention. The sensors can optionally be implanted and configured to communicate with the neurostimulation device or the sensors can be provided externally. In some examples, a sensor can include an interface that receives information from a patient or caregiver.

The present inventors have recognized that some autonomic auras may indicate pre-seizure signaling on the left and right vagus nerves, which provide the predominant innervation of abdominal viscera. The larger ratio of efferent-to-afferent nerves is in the right vagus, potentially providing a stronger signal path that can be sensed, for example, using implanted electrodes disposed at, near, or around a portion of the vagus nerve. Solutions discussed herein can include or use vagal electroneurogram information sensed from the left and/or right vagus nerves.

VNS can be used to provide epileptic seizure therapy prophylactically, or responsively such as using manual activation (e.g., via a patient-applied magnet) or in response to detected changes in heart rate or heart rate variability (HRV). While HRV may be driven by vagal signaling and is one manifestation of an autonomic aura, it may be insufficiently sensitive for patients who manifest other autonomic auras. The present inventors have recognized that information from an electroneurogram, such as a vagal electroneurogram (VENG), can be used for seizure detection. The present inventors have recognized that a solution to the seizure detection problem can include or use electroneurogram information. In an example, the solution includes systems or methods to detect vagus nerve-signaled autonomic aura manifestations for each patient's oncoming or in-process seizure. For each patient, pre-seizure and/or intra-seizure vagal signal characteristics can be monitored and seizure correlation patterns can be established. The patterns can then be used for detection of subsequent seizure events. Correlation patterns can include or use, but are not limited to, time or frequency domain features, such as power-spectral densities. The patterns can then be used to perform seizure detection based on the vagal signaling specific to a particular patient. The patient-specific correlation pattern can be identified automatically, such as using machine learning-based techniques, or can be identified by a clinician. In an example, other sensor information can be used together with VENG information to further enhance seizure detection accuracy. Such other sensor information can include heart rate information, motion or movement information, brain signal information, or other information, such as can be received from one or more implanted, body-worn, or external sensors. In other words, seizure detection, or detection of physiologic status that is known or learned to precede a seizure event, can be customized for individual patients, such as using physiologic parameters or parameter patterns that may be unique to each individual patient.

In an example, a solution to the seizure detection and therapy titration problems can include circuitry configured to sense patient-specific VENG information from electrodes that are implanted at, on, or around a portion of a patient's vagus nerve, such as a left branch or right branch of the vagus nerve. In an example, the circuitry comprises a portion of an implanted or implantable medical device. The circuitry can be configured to transfer stored or real-time VENG information to an analysis system, such as can be external to the patient or external to a VNS therapy device that is implanted in the patient. The analysis system can be configured to present the VENG information to a clinician, or can be configured to automatically perform various pattern detection routines or algorithms (e.g., machine learning-based algorithms) to identify correlations between the VENG information and patient seizure events. In an example, the analysis system (or clinician) can use VENG characteristic information such as, but not limited to, temporal, spectral, or phase information to identify characteristics that correlate with seizure events. In a particular example, patient-specific VENG information can be used as training data and can be associated, manually or automatically, with indications of seizures or seizure events, where the indications of seizures or seizure events are reported by the patient or the clinician. In an example, the analysis system, or a portion of the analysis system, can be included with the implantable medical device and the analysis can be performed on-board the device without transferring data externally to the device.

The present inventors have recognized that a personalized approach to seizure detection may be useful due in part to the variability in the manifestation of seizures across different patients or populations. Vagal electroneurograms (VENGs) capture the neural signals transmitted through the vagus nerve, which can contain specific patterns or characteristics indicative of an impending or ongoing seizure. These characteristics, however, may not be the same for all patients. For one patient, a particular VENG characteristic, or set of VENG characteristics, can reliably signal the onset of a seizure, while for another patient, a different set of characteristics can be more effective at signaling seizure onset. The patient-to-patient variability can be due to differences in individual patient physiology, the particular nature of each patient's seizure disorder, or other factors. That same patient-to-patient variability extends to other disorders as well, and accordingly the systems and methods discussed herein can be applied to disorders other than epilepsy or seizures.

To accommodate patient-specific variability, the systems and methods discussed herein can be configured to continuously, periodically, or intermittently monitor VENGs for the characteristics that have been identified as precursors to seizure events (or manifestations of other disorders) for a particular patient. The monitoring process is dynamic and can be adjusted as more data is collected, such as data about the patient's VENG activity and seizures, thereby allowing the system to refine its detection algorithms over time.

The systems discussed herein can respond to detecting a VENG characteristic that was previously identified as being correlated with a seizure event (or other disorder manifestation) for a patient. In an example, the system response can include generating or providing an alert or notification to the patient or their caregiver, enabling them to take appropriate precautions or to prepare for the possibility of a seizure. Such early warning systems can be useful in ensuring the safety of the patient, allowing for timely intervention and the mitigation of potential risks associated with seizures or other disorders. In an example, the system response can include automatically initiating or adjusting parameters of a neurostimulation therapy. For example, the response can include changing an intensity, frequency, duration, stimulation waveform, or other aspect of the therapy. The automated therapeutic response can help provide immediate and patient-specific intervention that can either prevent the seizure from occurring or lessen its severity.

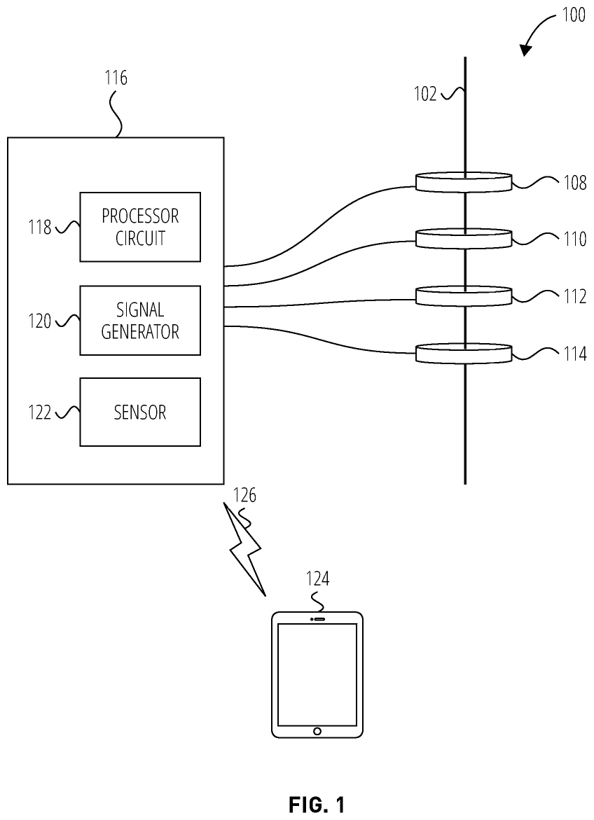

An illustrative (but non-restrictive) example, as shown in FIG. 1, includes a system 100 for providing neurostimulation to a vagus nerve 102, or vagus nerve stimulation (VNS). In an example, the system 100 can be configured to sense nerve activity or other electrical activity or motion. The example of the system 100 includes an implantable device 116 such as can comprise a processor circuit 118 and a signal generator 120. The processor circuit 118, or control circuit, can control operation of the signal generator 120 according to various therapy delivery algorithms or therapy signal-defining parameters. The signal generator 120 can be configured to generate neurostimulation signals or pulses according to parameters or instructions from the control circuit. In an example, the signal generator 120 includes independent current sources and controllers to enable independent and simultaneous output of multiple respective therapy signals. In various examples, some portions of the system 100 or the implantable device 116 can include wearable or other ambulatory devices.

In an example, the implantable device 116 comprises or is coupled to one or more physiologic status sensors that are configured to sense information about a patient. For example, the system can include a sensor 122. The sensor 122 can comprise a portion of the implantable device 116 or can be coupled to a lead that is coupled to the implantable device 116. In other examples, the sensor 122 can be an external sensor that is coupled to, or otherwise configured to receive information from, the patient. In an example, the sensor 122 comprises an accelerometer configured to sense motion information about the patient. In an example, the sensor 122 comprises one or more electrodes configured to sense electrical signals from the patient. In an example, the one or more electrodes can be implanted at or near a vagus nerve of the patient and can be configured to delivery electrical signals to, or receive electrical signals from, the vagus nerve. For example, the one or more electrodes can be configured to sense vagal electroneurogram information from the patient.

In an example, the system 100 includes an external device 124 that can communicate with the implantable device 116. The external device 124 can include a patient device or clinician device that is configured to receive information from, or provide information to, the implantable device 116. In an example, the external device 124 can include a display configured to receive and display data from the implantable device 116, including individual sensor data and seizure detection annotations. The system 100 or the external device 124 may calculate and display seizure burden, and/or display an event log. In an example, the external device 124 includes an interface that allows patients to confirm events and/or add comments or annotations to detected events.

For example, the external device 124 can be used to set one or more neurostimulation parameters for a neurostimulation therapy that is provided by the implantable device 116. In an example, the external device 124 can be used to report information to a patient or clinician about one or more therapies provided by the implantable device 116. In an example, the external device 124 includes one or more sensors that are configured to monitor physiologic or behavioral information about the patient.

The interaction between the external device 124 and the implantable device 116 is facilitated through a bidirectional communication link using a wireless coupling 126 that allows for the continuous exchange of data and commands between the two devices. The communication is established using wireless technology protocols that are specifically designed for medical devices, ensuring secure and reliable data transmission.

The external device 124 is equipped with various sensors, including a high-resolution camera, a microphone, and an accelerometer, which collect a wide array of physiological and environmental data, as described below. This data includes visual and audio records of the patient's movements, vocalizations, and surrounding environment, as well as quantitative measurements such as detected motion patterns and respiration rates. The collected data is then processed (e.g., at the external device 124, at the implantable device 116, or elsewhere) using advanced algorithms to identify potential seizure events or other disorder-related episodes.

In an example, the processor circuit 118 is programmed with a set of parameters that define thresholds for initiating or adjusting VNS therapy. Upon receiving a therapy-indicating signal from the external device 124, the processor circuit 118 can be configured to analyze its sensor data against these predefined parameters. If the data indicates that a seizure is occurring or imminent, the implantable device 116 adjusts the neurostimulation therapy parameters accordingly. This adjustment may involve changing the intensity, frequency, duration, or other characteristics of the electrical impulses delivered to the vagus nerve to provide an appropriate therapeutic response.

In an example, seizure detection and VNS can include or use one or two vagus nerve sensing electrodes (e.g., “recording cuff” or helical electrodes), such as located in different longitudinal positions along the cervical vagus region, relative to a stimulation site. Separate stimulating electrodes (e.g., an anode and a cathode) can be positioned to provide VNS. In the example of FIG. 1, the system 100 includes a first electrode 108, a separate second electrode 110, a separate third electrode 112, and a separate nth electrode 114 positioned at or near the vagus nerve 102. The various electrodes can be used in various combinations to provide an epilepsy therapy or to sense electroneurograms. In an example, the multiple electrodes comprise respective portions of a single lead, or multiple leads can be used, with each lead comprising one or more electrode.

The count and position of electrodes in the example of FIG. 1 is merely illustrative. For example, an implantable device can include circuitry for sensing (e.g., recording) neural activity (e.g., an action potential or compound action potential), along with circuitry for generating VNS signals. In such an example, an applied artificial intelligence approach can be implemented by the implant circuitry or the processor circuit 118. Such an approach can be used for detection of a seizure or for therapy titration, such as can be based on seizure detection.

In an example, the sensing electrodes and related circuitry can be separate from the stimulating electrodes and the sensing electrodes can be monitored by a separate unit (e.g., an external assembly) that can be used in an acute or temporary manner, such as supporting the implant procedure or implantable device configuration. For example, in the case that the sensing and stimulating electrodes are separate, the sensing electrode may be explanted acutely as a portion of a first procedure or soon after the first procedure. In yet another example, there could be three or more electrodes that could be configurable as either a stimulating electrode or a sensing electrode at any time. For example, two electrodes closest to a brain of a patient could be assigned as an anode and a cathode, respectively, and another electrode that is located more distally could be assigned as a sensing electrode to detect efferent nerve activation. As another illustration, two electrodes most distal to the brain could be assigned as an anode and a cathode, respectively, and an electrode more or most proximal to the brain could be assigned as a sensing electrode to detect afferent activity.

In an example, a lead can comprise one or more electrodes (e.g., the first electrode 108, the second electrode 110, the third electrode 112, and/or the nth electrode 114), and can optionally comprise a retention or affixation feature. The affixation feature can be provided at a proximal or distal end of the lead, or can be provided at an intermediate location along the length of the lead. The affixation feature can be electrically functional (e.g., comprising one or more electrodes for sensing or delivery of electrical neurostimulation) or electrically non-functional (e.g., without conductive materials or without electrodes). In some examples, an electrode can be coupled to, or integrated with, a retention feature.

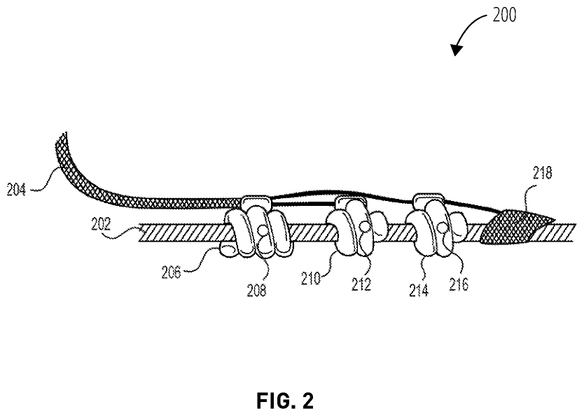

FIG. 2 illustrates generally an example of a first tripolar lead assembly 200 with a first retention feature 218. The first tripolar lead assembly 200 can be coupled to a stimulator circuit (e.g., in an implantable housing) and can be configured for implantation at a neural target, such as at the vagus nerve 102. The first tripolar lead assembly 200 can comprise a lead body 204 and one or more distal electrodes, anchors, or affixation features.

The first tripolar lead assembly 200 includes multiple helical anchors, and each of the anchors comprises a separately addressable electrode. For example, the first tripolar lead assembly 200 includes a first helical anchor 206 with a first electrode 208 (e.g., corresponding to the first electrode 108 from the example of the system 100), a second helical anchor 210 with a second electrode 212 (e.g., corresponding to the second electrode 110), and a third helical anchor 214 with a third electrode 216 (e.g., corresponding to the third electrode 112). Any one or more of the anchors can optionally comprise an array of multiple, separately-addressable electrodes. Each of the helical anchors can be configured to receive a respective portion of the vagus nerve 102 (or other nerve) and can be adjustable in size to accommodate variations in width of the vagus nerve 102 and other tissue. For ease of reference herein, the first electrode 208 can be referred to as “electrode A” or “A,” the second electrode 212 can be referred to as “electrode B” or “B,” and the third electrode 216 can be referred to as “electrode C” or “C.” Combinations or pairs of the electrodes used for electrostimulation can be referred to by letters, for example, electrode pair A-B can refer to one of the first electrode 208 and the second electrode 212 configured as an anode and the other of the electrodes configured as a cathode for use in an electrostimulation vector. In other examples, two or more of the electrodes can be electrically coupled to provide an anode or cathode for another electrostimulation vector. For example, the first electrode 208 and the second electrode 212 can be electrically coupled to provide an anode and the third electrode 216 can be used as a cathode. Other combinations can similarly be used to provide other electrostimulation vectors for neurostimulation therapy delivery or sensing. The various combinations can be used for respective different therapies or can be used together for one or multiple therapies.

In the example of FIG. 2, the electrodes are illustrated schematically as having discrete locations, however, other locations in, on, or around the helical anchors can be used. In an example, one or more of the electrodes can comprise a ring electrode or conductive ribbon that extends partially or entirely around a revolution of its respective helical anchor, such as to encircle the target tissue (e.g., the vagus nerve 102). Other configurations can similarly be used. In another example, multiple spaced apart electrodes can be provided on a single cuff or helical structure. Implanting a monolithic structure having multiple electrodes would generally be easier and faster for the physician, as compared to separate implantation of individual discrete electrode structures. In another example, multiple electrodes on one ring of a cuff can be used to selectively stimulate the target neural fibers. Use of multiple electrodes in an array, or a series of ring structures, can facilitate programmability of different spatial arrangements of neural activity sensing or stimulation (or both). In an example, an electrode array configuration can provide redundancy in case of loss of sensing or stimulation efficacy of a particular electrode. Such a multiple-electrode configuration can be used to provide sensing modalities or stimulation electrode configurations that can vary over time to maintain effectiveness of VNS therapy or VENG sensing or seizure detection.

In an example, the first retention feature 218 comprises a mesh or other structure. In the example of FIG. 2, the mesh structure can be coupled to a distal portion of the lead body 204 and configured to grow into tissue at, near, adjacent to, or around the vagus nerve 102 or other nerve tissue. In an example, additionally or alternatively to providing the first retention feature 218 at the distal portion of the lead body 204, one or more other instances of the first retention feature 218 can be coupled to a proximal or intermediate portion of the lead body 204.

The system 100 as shown and described herein can use a pattern detection or pattern recognition algorithm to identify characteristics of one or more physiologic signals that can be associated with one or more disorders. A pattern detection or recognition algorithm can include, but is not limited to, an artificial intelligence-based (e.g., machine learning) algorithm. In an example, the pattern detection or recognition algorithm can be defined manually (e.g., by a clinician or other system user) or can be computer-assisted.

For example, the system 100 can be configured to analyze one or more historical physiological signals (e.g., received from a particular patient, or a patient population) and identify specific patterns within these signals. The identified patterns can be associated with, or have a correlation to, various neurological and physiological events, conditions, or episodes (e.g., a condition with a duration or a series of related occurrences). In an example, an occurrence or status of the events or conditions can be reported by the patient or a clinician, or can be identified automatically using information from one or more sensors. After identifying one or more specific patterns, the system 100 can then apply a pattern recognition algorithm to recognize one or more of the patterns in other physiologic signal information, such as can be received from the same patient or a different patient. In an example, the same sensor or sensors can be used to receive the historical physiologic signal information (e.g., during a training or learning period) and to receive the other physiologic signal information (e.g., during a monitoring period).

For example, the system 100 can be configured to identify or recognize patterns that indicate an imminent or ongoing seizure event, or a precursor to such a seizure. In an example, the system 100 can be configured to identify or recognize patterns that are indicative of depression, which may include episodes of depression or other physiological signs that suggest the onset or progression of depressive states. In an example, the system 100 can be configured to identify or recognize patterns related to movement disorders, which can encompass a range of impairments such as, but not limited to, dysfunctional limb movement. In an example, the system 100 can be configured to identify or recognize patterns during stroke rehabilitation. The system 100 can be configured to identify or recognize patterns associated with other disorders or physiologic states as well.

In an example, the system 100 is configured to sense VENG information about a patient and identify VENG characteristics that can be associated with the one or more disorders. One or more electrodes can read vagus nerve activity (e.g., using electrical signal sensing) information and a processor (e.g., the processor circuit 118) can determine a profile of the neural activity of a patient. In an example, the VENG information can be received and processed together with one or more other signals from physiologic sensors or information reported from a patient. Various options and techniques for VENG processing are further discussed below.

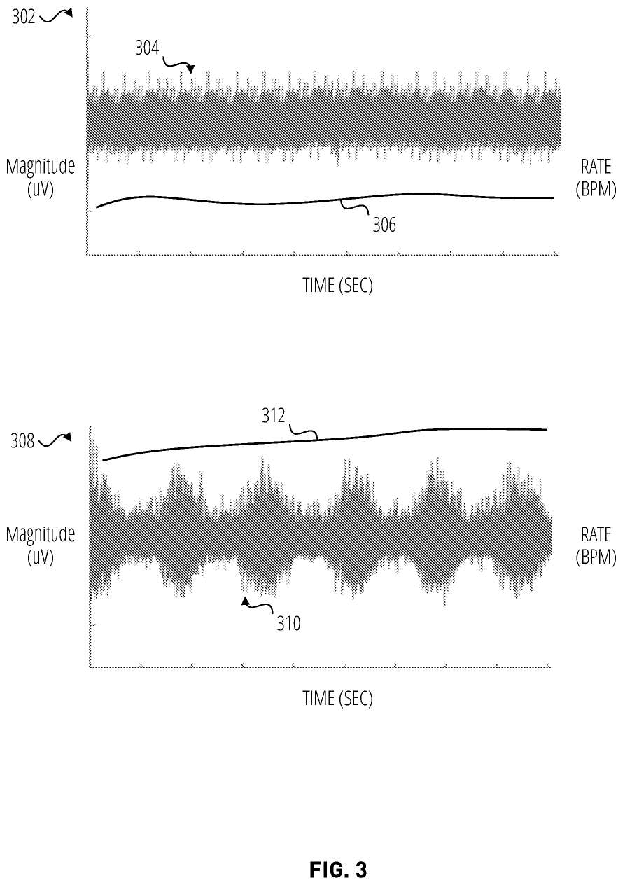

FIG. 3 illustrates generally examples of VENG information. The example of FIG. 3 includes a first physiologic status chart 302 and a second physiologic status chart 308. In an example, the charts represent physiologic status information from the same patient acquired using the same sensors during different times.

The first physiologic status chart 302 includes a first vagal electroneurogram 304 and a first heart rate signal 306. In an example, the first vagal electroneurogram 304 and the first heart rate signal 306 can be sensed concurrently, such as using respective different sensors. In an example, the information in the first physiologic status chart 302 illustrates generally an example of a baseline status for the patient. The baseline status can correspond to a reference or non-disordered state for the patient. For example, the baseline condition can correspond to a period of time when the patient is not experiencing a seizure, or when the patient is not experiencing a major depressive episode, etc. In the example of FIG. 3, the baseline status of the first vagal electroneurogram 304 indicates relatively constant, low-level vagal activity without significant spikes or irregularities, and the first heart rate signal 306 indicates a relatively low and steady heart rate. In an example, the system 100 can be configured to receive patient-reported or clinician-reported information about the patient's physiologic status, such as to confirm that the patient is not experiencing a seizure, depressive episode, or other event. For example, the patient-reported or clinician-reported information can be received using the external device 124 or using another sensor or device. The patient-reported or clinician-reported information can be received periodically, intermittently, or concurrently with receiving the VENG information or the heart rate information represented in the first physiologic status chart 302.

The second physiologic status chart 308 includes a second vagal electroneurogram 310 and a second heart rate signal 312. The second vagal electroneurogram 310 and the second heart rate signal 312 can be sensed concurrently. In an example, the information in the second physiologic status chart 308 illustrates generally an example of a disordered status for the patient. The disordered status can correspond to a particular disorder episode or event, such as a seizure event, or a seizure precursor. In the example of FIG. 3, the disordered status of the second vagal electroneurogram 310 manifests as a VENG signal with periodic spikes in neural activity, and the second heart rate signal 312 indicates an increasing and relatively high heart rate. In an example, patient-reported or clinician-reported information can be received, such as concurrently with receiving the sensor information represented in the second physiologic status chart 308, to confirm the occurrence of the episode or event experienced by the patient.

In an example, if the physiologic status information represented in the second physiologic status chart 308 is correlated with an episode or event, then the particular pattern represented in one or both of the physiologic signals can be identified (e.g., automatically by the processor circuit 118 or manually by a clinician). In the illustrated example, the particular pattern can be expressed in terms of characteristics of the physiologic signals, for example, periodic spikes in VENG signal magnitude (e.g., spikes exceeding 125% of baseline signal magnitude) occurring at a particular frequency (e.g., at 30-40 Hz). In an example, the particular pattern can be further expressed as including a heart rate characteristic, such as a heart rate at or above 150% of a baseline heart rate, or an increasing heart rate over a specified minimum duration. Following identification of the particular pattern, subsequent physiologic signal information (e.g., received from the same patient or a different patient) can be analyzed for the same pattern characteristics (e.g., VENG magnitude, spike frequency, heart rate, etc.). When the same characteristics are recognized in other physiologic signal data, then one or more responsive actions can occur. For example, the system 100 can initiate or titrate a neurostimulation therapy using the implantable device 116 to address the disorder associated with the pattern, or the system 100 can notify the patient or a caregiver (e.g., to notify the patient or caregiver that a seizure event is likely to be imminent). One, two, or more physiologic signals can be used to recognize patterns associated with a disorder or event. Generally, as shown and described herein, neural activity will be monitored (e.g., sensed electrically), either alone or in combination with other input variables.

The example of FIG. 3 discusses use of time domain VENG information. However, VENG information can be used in other forms, such as after transformation to the spectral domain. Transforming the VENG information into the spectral domain can allow extraction of features that may be indicative of disorders, disorder progression, or seizure events, among other things. The features can include changes in the signal content over time or can be used to determine specific patterns that correlate with disorder manifestations. Computer-implemented (e.g., machine learning-based) algorithms can use these features to classify segments of neural activity and detect potential disorder manifestations, such as alone or together with other physiologic status information that can be analyzed in the time domain, in the spectral domain, or using other signal processing techniques.

In an example, transforming VENG information to the spectral domain includes determining a power spectral density (PSD) for a portion of the VENG information. Power spectral density (PSD) is a measure of the power present in a signal as a function of frequency. That is, PSD can provide information about a distribution of power across various frequency components of the VENG signal. In an example, determining a PSD of an electroneurogram (ENG) includes multiple steps, including data acquisition, pre-processing, data transformation, and PSD calculation. Data acquisition can include recording an ENG signal using various electrodes, such as the electrodes coupled to the implantable device 116 of the system 100. The signal can be sampled at an adequate rate to capture the frequency content of interest. Pre-processing can include filtering (e.g., to remove noise or other unwanted frequency components, such as using band-pass filtering). Pre-processing can optionally include normalizing or applying a window function to reduce unwanted signal components. After pre-processing, the filtered signal can be transformed, such as by applying a Fast Fourier Transform (FFT) to convert the signal from the time domain to the frequency domain. The FFT decomposes the signal into its constituent frequencies and provides amplitude and phase information for each frequency component. In an example, a power spectral density can be computed by squaring the magnitude of the FFT results to obtain the power spectrum. The PSD is typically expressed in units of power per frequency (e.g., dB/Hz). For a continuous signal, the PSD is the square of the absolute value of the Fourier Transform divided by the signal length. For discrete signals, it can be normalized by the sampling rate. In an example, the signal processing can further include or use an average of the PSDs of multiple segments of the signal. The resulting PSD information can be analyzed such as to determine one or more patterns that can be associated with a patient disorder manifestation (e.g., a seizure event). In an example, a display (e.g., using the external device 124) can show a plot of the PSD against frequency to visualize the distribution of power across frequencies. Peaks in the PSD plot can indicate dominant frequencies or harmonics in the signal, which in turn can be used to identify or recognize patterns. That is, the PSD plot can be used to identify any patterns or characteristics that may be relevant to the physiological state being monitored, such as seizure activity. The specific methods and parameters used in each PSD calculation step can vary depending on the characteristics of the ENG signal, the equipment used, and the goals of the analysis.

Other physiologic and non-physiologic signals can be similarly analyzed for patterns associated with a disorder, disease, or progression of a disorder or disease. Examples of other physiologic signals can include, but are not limited to, signals comprising information about a patient heart rate, heart rate variability, blood pressure (e.g., systolic pressure, diastolic pressure, mean blood pressure, or contractility), respiration (e.g., respiration rate, phase or cycle information), electroencephalography (EEG), electrodermal activity or skin conductivity, temperature, odor, or other information. In an example, other inputs can be used for pattern analysis, including time of day information, geographic or atmospheric information, acoustic information, and more. For example, information about patient noises (e.g., vocalizations) or movements can be used for pattern identification and recognition. The other physiologic and/or non-physiologic information used for pattern identification and recognition can be received by a pattern analysis processor, such as the processor circuit 118 or other processor circuit comprising a portion of the system 100.

Machine learning, as a specialized form of pattern detection, can enhance the capabilities of the system 100 by enabling it to learn from historical data and improve its predictive accuracy over time. By providing a large dataset of physiological signals (e.g., sensed over time using the sensor 122 or other sensor(s) coupled to the implantable device 116 or configured to share information with the system 100) and known outcomes (e.g., patient-reported or clinician-reported confirmation of various episodes, events, disease progressions, etc.), machine learning algorithms can be trained to identify complex patterns that may not be readily apparent. Machine learning algorithms can include supervised learning, such as where the system is trained on labeled data, or unsupervised learning, where the system identifies patterns without pre-labeled outcomes.

In the context of seizure detection, machine learning models can be trained on a dataset comprising numerous instances of pre-seizure and seizure VENGs, such as along with other corresponding physiologic status-indicating signals (e.g., heart rate signals, etc.). The model can learn to discern the characteristics that differentiate between normal physiologic (e.g., neural) activity and the onset of a seizure. This can involve identifying specific frequency spikes in the VENG signal or particular heart rate patterns that have been historically associated with seizures. After training, the system 100 can monitor a patient's real-time data and provide an alert when it detects a pattern that suggests a seizure event or that a seizure may be imminent.

In an example, a machine learning algorithm can be optimized by identifying the feature or features that are particularly informative for the prediction task. For example, in addition to raw signal data, features such as the variability of a signal characteristic (e.g., amplitude, or a regularity of frequency peaks, etc.) can be used as inputs. In an example, a machine learning algorithm can be designed to incorporate feedback loops, allowing the system to continuously learn and adapt to each patient's unique physiological patterns. This adaptability allows for customized, patient-specific applications and ensures that the system 100 remains sensitive to the individual's changing physiological state and maintains high accuracy in pattern recognition over time.

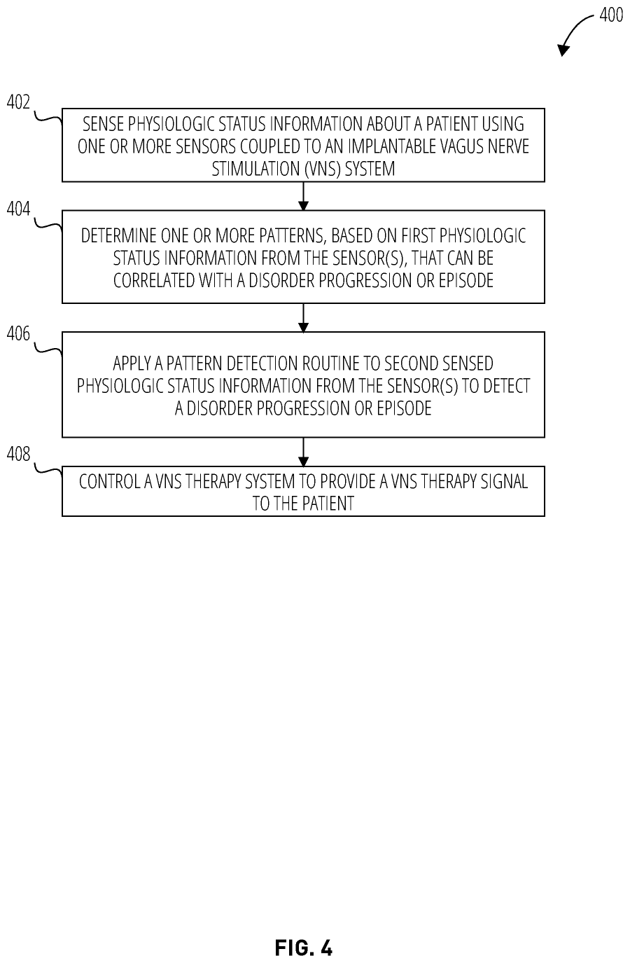

FIG. 4 illustrates an example of a first method 400 for using a vagus nerve stimulation (VNS) system to provide a VNS therapy to a patient. Although the example first method 400 depicts a particular sequence of operations, the sequence may be altered without departing from the scope of the present disclosure. For example, some of the operations depicted may be performed in parallel or in a different sequence that does not materially affect the function of the first method 400. In other examples, different components of an example device or system that implements the first method 400 may perform functions at substantially the same time or in a specific sequence. In an example, some or all of the operations of the first method 400 can be performed using components of the system 100. In an example, one or more of the data analysis-related operations of the first method 400, such as can include pattern identification or recognition, can be performed using a remote diagnostic system.

At operation 402, the first method 400 includes sensing physiologic status information about a patient using one or more sensors. The sensors can be coupled to an implantable vagus nerve stimulation (VNS) system. In an example, operation 402 includes receiving physiologic status information from a patient using one or more sensors (e.g., the sensor 122) that are coupled to, or comprise a portion of, the implantable device 116 of the system 100. In an example, the one or more sensors are configured to monitor various respective physiologic parameters that can include, but are not limited to, vagal electroneurogram (VENG) information, heart rate, heart rate variability, blood pressure, and respiration information, among other things. In an example, operation 402 includes receiving audio or visual (e.g., image) information about the patient using a camera (e.g., comprising an example of a sensor 122). The audio or visual information can be analyzed using image processing to identify patient characteristics, movements, etc., that may be indicative of an adverse event or patient disorder progression. In an example, data received or collected by the one or more sensors can be used as an input for a disorder detection algorithm or therapy titration algorithm. In an example, the detection algorithm can be a machine learning-based model or algorithm.

At operation 404, the first method 400 includes determining one or more patterns based on first physiologic status information received from the one or more sensors at operation 402. The operation 404 can include determining one or more patterns that can be correlated with a disorder progression or episode. For example, operation 404 can include determining one or more patterns, based on first physiologic status information from the sensor(s), that can be correlated with an in-process seizure event or an imminent seizure event. In another example, operation 404 can include determining one or more patterns that can be correlated with depression, movement disorders, or other disorders.

At operation 406, the first method 400 includes applying a pattern detection routine (e.g., manually, or using a processor or computer) to second sensed physiologic status information from the patient. In an example, the second sensed physiologic status information is sensed using the same sensor or sensors used at operation 402, and the second sensed physiologic status information is received subsequently to the first physiologic status information used to determine the pattern(s) at operation 404. The operation 406 can include applying the pattern detection routine to detect progression of a disorder, or to detect an episode associated with the disorder. For example, operation 406 can include applying the pattern detection routine to detect a seizure event, or to determine a likelihood that a seizure event is imminent.

In an example, operation 406 includes applying the pattern detection routine substantially in real-time with acquisition of the second sensed physiologic status information to achieve early detection (e.g., of seizures) or timely prediction of disorder episodes (e.g., imminent seizure events). In an example, the pattern detection routine performs pattern recognition using the incoming sensor data to determine whether the physiological signals align with or include the characteristics of a particular predefined pattern (e.g., corresponding to a seizure or other disorder). If the system detects a pattern match or identifies a high probability of a pattern match, then it triggers a specified response.

At operation 408, the first method 400 includes controlling the VNS system to provide a VNS therapy signal to the patient. For example, operation 408 can include using the VNS system to treat a seizure event. In response to a result of the analysis performed at operation 408, the VNS system can be activated to generate and provide a therapeutic neurostimulation signal to the vagus nerve. The parameters of this VNS therapy signal, such as intensity, frequency, duty cycle, waveform, and duration, among other parameters, can be automatically adjusted to the patient's immediate needs to effectively treat the particular identified disorder. Accordingly, timely and patient-specific intervention can be provided. In the case of seizure detection, early intervention can help prevent a seizure from occurring, reduce its severity, or shorten its duration.

In an example, following operation 406 or operation 408, the pattern detection routine can be iteratively refined to further enhance system efficacy over time. The system can be configured to continuously adapt to changes in the patient's physiologic manifestation of a disorder (e.g., a seizure event) or to changes in the patient's response to the VNS therapy delivered at operation 408.

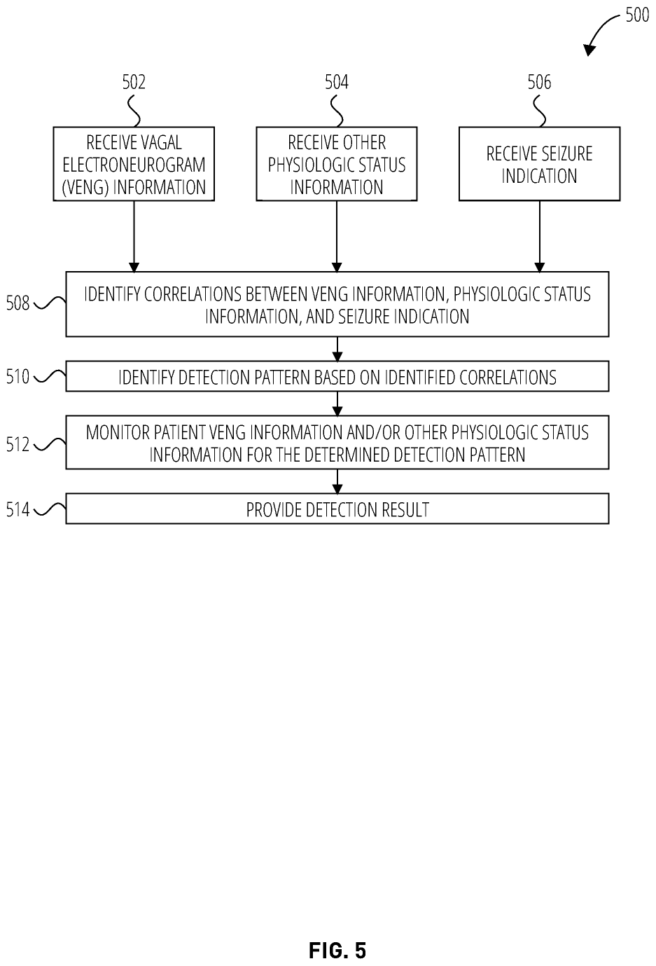

FIG. 5 illustrates an example of a second method 500 that can include pattern identification and recognition for use in seizure detection. Although the example second method 500 depicts a particular sequence of operations, the sequence may be altered without departing from the scope of the present disclosure. For example, some of the operations depicted may be performed in parallel or in a different sequence that does not materially affect the function of the second method 500. In other examples, different components of an example device or system that implements the second method 500 may perform functions at substantially the same time or in a specific sequence. In other examples, the same or similar method can be applied for pattern identification and/or recognition of manifestations of other disorders or diseases, such as including but not limited to movement-related disorders, depression, stroke rehabilitation, or others. In an example, some or all of the operations of the second method 500 can be performed using components of the system 100. In an example, one or more of the data analysis-related operations of the second method 500, such as can include pattern identification or recognition, can be performed using a remote diagnostic system.

The example of the second method 500 can begin with receiving various patient-specific or patient population-specific information. For example, the second method 500 can include receiving vagal electroneurogram (VENG) information at operation 502, receiving other physiologic status information at operation 504, and receiving a seizure indication at operation 506. In an example, operation 504 and/or operation 506 can include receiving information from a sensor (e.g., one or more of the sensors 122) or receiving information reported by a patient or clinician.

In an example, operation 504 includes receiving patient-reported information about gastrointestinal sensations experienced by the patient, such as can include information about discomfort in the abdominal or periumbilical area that can be static, rise to the chest and throat, or descend into the lower abdominal region. In an example, operation 504 includes receiving patient-reported information about genitourinary sensations experienced by the patient, such as can include genital sensations, urinary urges, and other sensations. In an example, operation 504 includes receiving patient-reported information about cutaneous sensations experienced by the patient, such as can include feelings of warmth or cold. In an example, operation 504 includes receiving other autonomic aura-related information from the patient, including but not limited to information about sensations of nausea or pain. These auras may be indicative of pre-seizure signaling on the left and right vagus nerves, which provide the predominant innervation of abdominal viscera. The larger ratio of efferent-to-afferent nerves is in the right vagus, potentially providing a stronger signal path that can be sensed and analyzed, such as using the systems and methods discussed herein. In an example, operation 504 includes receiving image-based information from a camera that shows, or provides information about, a patient status. The image-based information can be processed by an image recognition algorithm to provide information about patient motion or movement that may be indicative of a seizure or pre-seizure aura.

In an example, the information received at operation 502, operation 504, and/or operation 506 can be received continuously, intermittently, periodically, or at other intervals. A first portion, or training portion, of the information received at operation 502, operation 504, and/or operation 506 can correspond to a training period and can be used to establish one or more patterns or models for seizure identification or prediction. A second portion, or monitoring portion, of the information received at operation 502, operation 504, and/or operation 506 can correspond to a monitoring period during which the patterns or models can be used for seizure identification or prediction. In an example, the patterns or models can be updated or tuned using information from the monitoring portion.

At operation 508, the second method 500 includes identifying correlations between the various inputs received at operation 502, operation 504, and/or operation 506. For example, operation 508 can include identifying correlations between VENG information, other physiologic status information (e.g., heart rate information, patient movement or activity level information, etc.), and a seizure indication, such as can be received during a training period. The seizure indication can include a patient-reported indication of a seizure.

In an example, operation 508 can include identifying temporal differences between various features of the inputs. For example, receiving the seizure indication at operation 506 can occur after signals of interest are received at operation 502 and/or operation 504. For example, the VENG information received at operation 502 may show a series of neural activity spikes that precede a seizure. By analyzing the timing of these spikes in relation to the seizure indication received at operation 506, the system can construct a timeline of physiologic status-indicating signal characteristics that lead up to a seizure. Similarly, the other physiological signals received at operation 504 may exhibit changes that occur in a specific order or within a particular time window before a seizure. By identifying these temporal patterns, the system can recognize early warning signs or precursors of a seizure. For example, a gradual increase in heart rate that consistently occurs several minutes before a seizure can be a temporal feature for the system to identify and use in its pattern recognition.

The temporal differences identified in operation 508 are not limited to pre-seizure indicators. They can also include the duration of the seizure itself, as well as post-seizure physiological changes. Understanding the full temporal context of seizures helps in creating a comprehensive model of seizure dynamics that can be used for future seizure detection.

At operation 510, the second method 500 includes identifying a detection pattern that is based on the identified correlations from operation 508. The operation 510 can include using data processing and analysis to parse the raw data and identified correlations into a simplified yet effective pattern (or patterns) that can be recognized while processing later-received sensor data. In an example, the detection pattern includes a set of criteria or a profile that describes characteristics of pre-seizure or intra-seizure physiologic signal behavior. For example, the detection pattern can include or use specific VENG signal characteristics and heart rate change characteristics which have been statistically linked to the onset or occurrence of seizures (e.g., for the patient, or for a population of patients). In an example, operation 510 can include identifying a pre-seizure signal pattern, an intra-seizure signal pattern, or both.

At operation 512, the second method 500 includes ongoing patient monitoring for recognition of a pattern, such as the detection pattern identified at operation 510. The operation 512 can include monitoring the patient VENG information, or the other physiologic status information, for the identified detection pattern. This continuous monitoring enables proactive management of seizure disorders. The system monitors for the detection pattern (e.g., a pattern identified at operation 510) within the incoming physiological data. If the pattern is recognized, indicating a potential seizure, then the system can trigger an alert or initiate a predefined therapy response protocol, such as to begin or update a VNS therapy.

At operation 514, the second method 500 includes providing a detection result. In an example, the detection result can include a notification that the detection pattern has been recognized, such as can suggest a seizure is occurring or imminent. In an example, a detection result that indicates a seizure can trigger the therapy response protocol. In an example, the detection result can include recommendations for immediate actions, such as initiating therapeutic interventions or alerting emergency services.

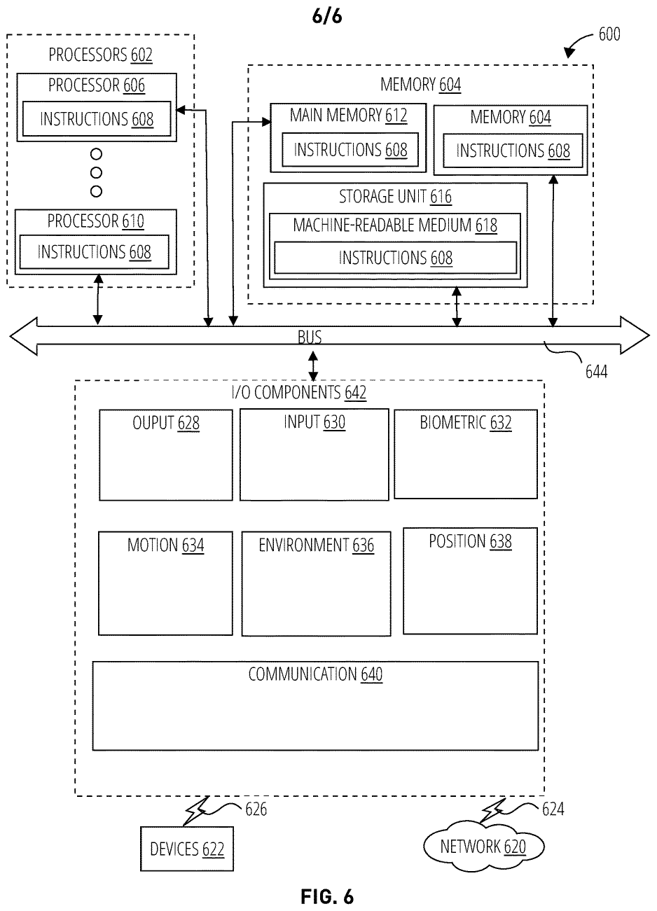

FIG. 6 illustrates generally an example of a machine in the form of a computer system within which a set of instructions may be executed for causing the machine to perform any one or more of the methodologies discussed herein, according to an example embodiment. FIG. 6 is a diagrammatic representation of a machine 600 within which instructions 608 (e.g., software, a program, an application, an applet, an app, or other executable code) for causing the machine 600 to perform any one or more of the methodologies discussed herein may be executed. In an example, the implantable device 116 or the external device 124, or one or more other components or devices in communication with the implantable device 116 and/or the external device 124, can comprise an example of the machine 600.

In an example, the instructions 608 may cause the machine 600 to execute any one or more of the methods, controls, therapy algorithms, signal generation routines, or other processes described herein. The instructions 608 transform the general, non-programmed machine 600 into a particular machine 600 programmed to carry out the described and illustrated functions in the manner described. The machine 600 may operate as a standalone device or may be coupled (e.g., networked) to other machines. In a networked deployment, the machine 600 may operate in the capacity of a server machine or a client machine in a server-client network environment, or as a peer machine in a peer-to-peer (or distributed) network environment. The machine 600 can comprise, but is not limited to, various systems or devices that can communicate with the components of the system 100, such as can include a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 608, sequentially or otherwise, that specify actions to be taken by the machine 600. Further, while only a single machine 600 is illustrated, the term “machine” shall also be taken to include a collection of machines that individually or jointly execute the instructions 608 to perform any one or more of the methodologies discussed herein.

The machine 600 may include processors 602, memory 604, and I/O components 642, which may be configured to communicate with each other via a bus 644. In an example embodiment, the processors 602 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 606 and a processor 610 that execute the instructions 608. The term “processor” is intended to optionally include multi-core processors that may comprise two or more independent processors (sometimes referred to as “cores”) that may execute instructions contemporaneously. Although FIG. 6 shows multiple processors 602, the machine 600 may include a single processor with a single core, a single processor with multiple cores (e.g., a multi-core processor), multiple processors with a single core, multiple processors with multiples cores, or any combination thereof.

The memory 604 includes a main memory 612, a static memory 614, and a storage unit 616, both accessible to the processors 602 via the bus 644. The main memory 604, the static memory 614, and storage unit 616 store the instructions 608 embodying any one or more of the methodologies or functions described herein. The instructions 608 may also reside, completely or partially, within the main memory 612, within the static memory 614, within a machine-readable medium 618 within the storage unit 616, within at least one of the processors 602 (e.g., within the processor's cache memory), or any suitable combination thereof, during execution thereof by the machine 600.

The I/O components 642 may include a variety of components to receive input, provide output, produce output, transmit information, exchange information, capture measurements, and so on. The specific I/O components 642 that are included in a particular machine will depend on the type of machine. For example, portable machines such as device programmers or mobile phones may include a touch input device or other such input mechanisms, while a headless server machine will likely not include such a touch input device. It will be appreciated that the I/O components 642 may include other components that are not shown in FIG. 6. In various example embodiments, the I/O components 642 may include output components 628 (e.g., comprising the signal generator 120) and input components 630 (e.g., one or more electrodes or other sensors). In an example, the 1/O components 642 can comprise a magnet or magnetic relay switch configured to be responsive to the presence or proximity of the magnet. The output components 628 may include pictorial, graphical, or visual components (e.g., a display such as a plasma display panel (PDP), a light emitting diode (LED) display, a liquid crystal display (LCD), a projector, or a cathode ray tube (CRT)) such as can be used by external device 124, or other interfaces that can be configured to display therapy parameter, intensity or effectiveness metrics, among other information. The output components 628 can include acoustic components (e.g., speakers), haptic components (e.g., a vibratory motor, resistance mechanisms), other signal generators, and so forth. The input components 630 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), physiologic sensor components, and the like.

In further example embodiments, the I/O components 642 may include biometric components 632, motion components 634, environmental components 636, or position components 638, among others. For example, the biometric components 632 can include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 634 can include an acceleration sensor (e.g., an accelerometer), gravitation sensor components, rotation sensor components (e.g., a gyroscope), or similar. The environmental components 636 can include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment, such as may contribute to the onset of seizures. The position components 638 can include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a variety of technologies. The I/O components 642 further include communication components 640 operable to couple the machine 600 to a network 620 or other devices 622 via a coupling 624 and a coupling 626, respectively. For example, the communication components 640 may include a network interface component or another suitable device to interface with the network 620. In further examples, the communication components 640 may include wired communication components, wireless communication components, cellular communication components, Near Field Communication (NFC) components, Bluetooth components, or Wi-Fi components, among others. The devices 622 may be another machine or any of a wide variety of peripheral devices such as can include other implantable or external devices.

The various memories (e.g., memory 604, main memory 612, static memory 614, and/or memory of the processors 602) and/or storage unit 616 can store one or more sets of instructions and data structures (e.g., software) embodying or used by any one or more of the methodologies or functions described herein. These instructions (e.g., the instructions 608), when executed by processors 602, cause various operations to implement the disclosed embodiments, including various neuromodulation or neurostimulation therapies or functions supportive thereof.

To better illustrate the systems and methods described herein, such as can be used to optimize a VNS therapy using physiologic status or signal pattern recognition, a non-limiting set of Example embodiments are set forth below as numerically identified Examples.

Example 1 is a seizure management system comprising: a first sensor configured to receive electrical signal information from a vagus nerve of a patient; and a processor circuit configured to: identify, in a training portion of the electrical signal information from the vagus nerve, one or more pre-seizure or intra-seizure signal patterns; monitor a monitoring portion of the electrical signal information from the vagus nerve for signal characteristics that correspond to the identified pre-seizure or intra-seizure signal patterns; and, responsive to recognizing, in the monitoring portion of the electrical signal information, signal characteristics that correspond to the identified pre-seizure or intra-seizure signal patterns, at least one of titrating a vagal nerve stimulation (VNS) therapy for the patient or notifying the patient or a caregiver about the identified pre-seizure or intra-seizure signal patterns.

In Example 2, the subject matter of Example 1 can optionally include an implantable device configured to provide the VNS therapy to the patient.

In Example 3, the subject matter of Example 2 can optionally include the implantable device comprises the first sensor.

In Example 4, the subject matter of Example 3 can optionally include the implantable device comprises the processor circuit.

In Example 5, the subject matter of any one or more of Examples 1-4 can optionally include the electrical signal information comprises vagal electroneurogram information, and the processor circuit is configured to identify the pre-seizure or intra-seizure signal patterns using the electroneurogram information.

In Example 6, the subject matter of any one or more of Examples 1-5 can optionally include a second sensor configured to receive other physiologic status information from or about the patient. In Example 6, the processor circuit can be configured to use the training portion of the electrical signal information from the vagus nerve together with the other physiologic status information from the second sensor to identify the pre-seizure or intra-seizure signal patterns.

In Example 7, the subject matter of Example 6 can optionally include the second sensor is configured to receive the other physiologic status information concurrently (e.g., simultaneously, contemporaneously, etc.) with receipt of the electrical signal information by the first sensor.

In Example 8, the subject matter of Example 7 can optionally include the processor circuit is configured to monitor the monitoring portion of the electrical signal information together with a monitoring portion of the other physiologic status information received from the second sensor for correspondence with the identified pre-seizure or intra-seizure signal patterns.

In Example 9, the subject matter of Example 8 can optionally include the second sensor comprises a cardiac activity sensor (e.g., an accelerometer, a pressure sensor, a thoracic impedance sensor, an internal or external electrical activity sensor, etc.) configured to provide physiologic status information about a heart rate or heart rate variability or heart palpitation of the patient.

In Example 10, the subject matter of any one or more of Examples 8-9 can optionally include the second sensor comprises an interface configured to receive patient-reported information about gastrointestinal sensations experienced by the patient.

In Example 11, the subject matter of any one or more of Examples 8-10 can optionally include the second sensor comprises an interface configured to receive patient-reported information about genitourinary sensations experienced by the patient.

In Example 12, the subject matter of any one or more of Examples 8-11 can optionally include the second sensor comprises an interface configured to receive patient-reported information about cutaneous sensations experienced by the patient.

In Example 13, the subject matter of any one or more of Examples 8-12 can optionally include the second sensor comprises a camera configured to receive image information about the patient.

In Example 14, the subject matter of Example 13 can optionally include an image processor circuit configured to analyze the image information from the camera to identify information about a patient movement or behavior that correlates with a previously-identified pre-seizure or intra-seizure patient movement.

In Example 15, the subject matter of any one or more of Examples 1-14 can optionally include the processor circuit is configured to identify the pre-seizure or intra-seizure signal patterns using seizure event information received from or about the patient.

Example 16 is a method comprising: sensing physiologic status information about a patient using a sensor coupled to an implantable vagus nerve stimulation (VNS) system; applying, using a processor circuit, a pattern detection algorithm to the physiologic status information to detect a seizure event or to determine that a seizure event is imminent for the patient; and in response to a result from the pattern detection algorithm indicating the seizure event was detected or is imminent, controlling a signal generator of the VNS system to provide a VNS therapy signal to the patient to treat the seizure event.

In Example 17, the subject matter of Example 16 can optionally include applying the pattern detection algorithm by applying a machine learning-based algorithm to analyze the physiologic status information and detect the seizure event or determine that the seizure event is imminent.

In Example 18, the subject matter of any one or more of Examples 16-17 can optionally include identifying patterns to be detected by the pattern detection algorithm, wherein the patterns are based on the physiologic status information about the patient received from the sensor.

In Example 19, the subject matter of Example 18 can optionally include identifying the patterns to be detected using vagal electroneurogram information about the patient.

In Example 20, the subject matter of Example 19 can optionally include sensing the vagal electroneurogram information about the patient using one or more electrodes coupled to the implantable VNS system and disposed at or near a vagus nerve of the patient.

In Example 21, the subject matter of Example 20 can optionally include sensing heart rate information about the patient. In Example 21, identifying the patterns to be detected can include identifying correlations between characteristics of the vagal electroneurogram information and characteristics of the heart rate information.

In Example 22, the subject matter of any one or more of Examples 16-21 can optionally include sensing the physiologic status information including receiving information about cardiac or respiratory characteristics of the patient.

In Example 23, the subject matter of any one or more of Examples 16-22 can optionally include receiving patient-reported information about gastrointestinal, genitourinary, and/or cutaneous sensations experienced by the patient. In Example 23, applying the pattern detection algorithm can include using the sensed physiologic status information together with the patient-reported information to detect the seizure event or to determine that the seizure event is imminent for the patient.

Example 24 is a seizure management system comprising: an implantable vagus nerve stimulation (VNS) system configured for implantation in a patient, the VNS system comprising a signal generator circuit and a sensor circuit; an external interface device; and a processor circuit configured to apply a machine learning-based model to information received from the sensor circuit to detect a seizure event or determine a likelihood that a seizure event is imminent for the patient; wherein the machine learning-based model is trained using information about the patient received from the sensor circuit and using patient-reported or clinician-reported information about a seizure event received from the external interface device. In Example 24, in response to the processor circuit detecting the seizure event or determining that a seizure event is imminent based on the determined likelihood, the processor circuit is configured to control the signal generator circuit to generate a VNS therapy signal.

In Example 25, the subject matter of Example 24 can optionally include the VNS system comprises a first electrode configured for implantation at or near a first neural target in the patient, and the first electrode is configured to provide the VNS therapy signal from the signal generator circuit to the first neural target.

In Example 26, the subject matter of any one or more of Examples 24-25 can optionally include the VNS system comprises a second electrode configured for implantation at or near a second neural target in the patient, wherein the second electrode is configured to receive electrical activity information from a vagus nerve of the patient, and wherein the sensor circuit is coupled to the second electrode.

In Example 27, the subject matter of any one or more of Examples 24-26 can optionally include the sensor circuit is configured to sense vagal electroneurogram (VENG) information from the patient.

In Example 28, the subject matter of Example 27 can optionally include the machine learning-based model is trained using historical VENG information from the patient.

In Example 29, the subject matter of Example 28 can optionally include the machine learning-based model is configured to identify a particular pattern in the historical VENG information that correlates with prior patient seizures, and wherein the processor circuit is configured to monitor subsequent VENG information from the patient for the same particular pattern.

In Example 30, the subject matter of Example 29 can optionally include, in response to recognizing the same particular pattern in the subsequent VENG information, using the processor circuit to provide an alert to the patient or a caregiver.

In Example 31, the subject matter of any one or more of Examples 29-30 can optionally include the sensor circuit is configured to receive information about a patient heart rate. In Example 31, the machine learning-based model is configured to identify the particular pattern that correlates with prior patient seizures based on the information about the patient heart rate and the historical VENG information.

In Example 32, the subject matter of any one or more of Examples 24-31 can optionally include the machine learning-based model is trained using historical information about multiple physiologic parameters of the patient.

In Example 33, the subject matter of Example 32 can optionally include the multiple physiologic parameters of the patient comprises vagal electroneurogram information.

In Example 34, the subject matter of Example 33 can optionally include the machine learning-based model is trained using power spectral density information determined from the vagal electroneurogram information.

In Example 35, the subject matter of Example 34 can optionally include the processor circuit configured to determine the power spectral density information.

In Example 36, the subject matter of Example 35 can optionally include the processor circuit comprises a portion of a remote diagnostic system.

In Example 37, the subject matter of any one or more of Examples 33-36 can optionally include the machine learning-based model is trained using temporal, spectral, or phase characteristics determined from the vagal electroneurogram information.

In Example 38, the subject matter of any one or more of Examples 33-37 can optionally include the vagal electroneurogram information is received from other than the implantable VNS system.

In Example 39, the subject matter of any one or more of Examples 33-38 can optionally include the vagal electroneurogram information includes information about a left vagus nerve, a right vagus nerve, or both the left and right vagus nerves.

In Example 40, the subject matter of any one or more of Examples 33-39 can optionally include the multiple physiologic parameters of the patient further comprises heart rate information or heart rate variability information about the patient.

In Example 41, the subject matter of Example 40 can optionally include using the sensor circuit of the implantable VNS system to receive the information about the multiple physiologic parameters.

In Example 42, the subject matter of any one or more of Examples 33-41 can optionally include or use heart palpitation information about the patient as one of the physiologic parameters.

In Example 43, the subject matter of any one or more of Examples 33-42 can optionally include or use respiratory information about the patient as one of the physiologic parameters.

In Example 44, the subject matter of any one or more of Examples 33-43 can optionally include or use patient-reported information about gastrointestinal, genitourinary, and/or cutaneous sensations experienced by the patient as one of the physiologic parameters.

In Example 45, the subject matter of any one or more of Examples 24-44 can optionally include the machine learning-based model is trained using vagal electroneurogram information from multiple patients.

In Example 46, the subject matter of any one or more of Examples 24-45 can optionally include the implantable VNS system comprises the processor circuit.

In Example 47, the subject matter of any one or more of Examples 24-46 can optionally include the external interface device comprises the processor circuit.

In Example 48, the subject matter of any one or more of Examples 24-47 can optionally include the processor circuit is configured to apply the machine learning-based model to determine whether one or more patterns in physiologic information received from the sensor circuit indicates the seizure event or indicates an increased likelihood that a seizure event is imminent.

Example 49 is at least one machine-readable medium including instructions that, when executed by processing circuitry, cause the processing circuitry to perform operations to implement of any of Examples 1-48.

Each of these non-limiting examples can stand on its own, or can be combined in various permutations or combinations with one or more of the other examples.

This detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. The present inventors contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.”

In the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like.

Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following statements (aspects) are hereby incorporated into the Detailed Description as examples or embodiments, with each standing on its own as a separate embodiment, and it is contemplated that such embodiments can be combined with each other in various combinations or permutations.

1. A seizure management system comprising:
a first sensor configured to receive electrical signal information from a vagus nerve of a patient, wherein the electrical signal information comprises vagal electroneurogram (VENG) information; and
a processor circuit configured to:
determine power spectral density information from the VENG information;
identify, in a training portion of the power spectral density information, one or more pre-seizure or intra-seizure signal patterns;
monitor a monitoring portion of the power spectral density information for signal characteristics that correspond to the identified pre-seizure or intra-seizure signal patterns that are specific to the patient; and
responsive to recognizing, in the monitoring portion of the electrical signal information, signal characteristics that correspond to the identified pre-seizure or intra-seizure signal patterns, at least one of titrating a vagal nerve stimulation (VNS) therapy for the patient or notifying the patient or a caregiver about the identified pre-seizure or intra-seizure signal patterns.

2. The seizure management system of claim 1, comprising an implantable device configured to provide the VNS therapy to the patient, wherein the implantable device comprises the first sensor and the processor circuit.

3-5. (canceled)

6. The seizure management system of claim 1, comprising a second sensor configured to receive other physiologic status information from or about the patient;
wherein the processor circuit is configured to use the training portion of the power spectral density information together with the other physiologic status information from the second sensor to identify the pre-seizure or intra-seizure signal patterns.

7. The seizure management system of claim 6, wherein the second sensor is configured to receive the other physiologic status information concurrently with receipt of the electrical signal information by the first sensor, and wherein the processor circuit is configured to monitor the monitoring portion of the power spectral density information together with a monitoring portion of the other physiologic status information received from the second sensor for correspondence with the identified pre-seizure or intra-seizure signal patterns.

8. (canceled)

9. The seizure management system of claim 7, wherein the second sensor comprises a cardiac activity sensor configured to provide physiologic status information about a heart rate or heart rate variability or heart palpitation of the patient.

10. The seizure management system of claim 7, wherein the second sensor comprises an interface configured to receive patient-reported information about gastrointestinal sensations, genitourinary sensation, and/or cutaneous sensations experienced by the patient.

11-12. (canceled)

13. The seizure management system of claim 7, wherein the second sensor comprises a camera configured to receive image information about the patient, and wherein the system comprises an image processor circuit configured to analyze the image information from the camera to identify information about a patient movement or behavior that correlates with pre-seizure or intra-seizure patient movement.

14. (canceled)

15. The seizure management system of claim 1, wherein the processor circuit is configured to identify the pre-seizure or intra-seizure signal patterns using seizure event information received from or about the patient.

16. A method comprising:
sensing physiologic status information about a patient using a sensor coupled to an implantable vagus nerve stimulation (VNS) system, wherein the physiologic status information includes vagal electroneurogram (VENG) information about the patient;
determining, using a processor circuit, power spectral density information from the VENG information;
applying, using a processor circuit, a pattern detection algorithm to the physiologic status information to detect a seizure event or to determine that a seizure event is imminent for the patient; and
in response to a result from the pattern detection algorithm indicating the seizure event was detected or is imminent, controlling a signal generator of the VNS system to provide a VNS therapy signal to the patient to treat the seizure event.

17. The method of claim 16, wherein applying the pattern detection algorithm includes applying a machine learning-based algorithm to analyze the power spectral density information and detect the seizure event or determine that the seizure event is imminent.

18-19. (canceled)

20. The method of claim 16, comprising:
sensing the vagal electroneurogram information about the patient using one or more electrodes coupled to the implantable VNS system and disposed at or near a vagus nerve of the patient; and
sensing heart rate information about the patient;
wherein identifying the patterns to be detected includes identifying correlations between characteristics of the vagal electroneurogram information and characteristics of the heart rate information.

21. (canceled)

22. The method of claim 16, wherein sensing the physiologic status information includes receiving information about cardiac or respiratory characteristics of the patient.

23. The method of claim 16, comprising receiving patient-reported information about gastrointestinal, genitourinary, and/or cutaneous sensations experienced by the patient;
wherein applying the pattern detection algorithm includes using the sensed physiologic status information together with the patient-reported information to detect the seizure event or to determine that the seizure event is imminent for the patient.

24. A seizure management system comprising:
an implantable vagus nerve stimulation (VNS) system configured for implantation in a patient, the VNS system comprising a signal generator circuit and a sensor circuit;
an external interface device; and
a processor circuit configured to apply a machine learning-based model to information received from the sensor circuit to detect a seizure event or determine a likelihood that a seizure event is imminent for the patient;
wherein the machine learning-based model is trained using historical information about multiple physiologic parameters of the patient received from the sensor circuit and using patient-reported or clinician-reported information about a seizure event received from the external interface device, wherein the multiple physiologic parameters of the patient comprise vagal electroneurogram (VENG) information, and wherein the machine learning-based model is trained using power spectral density information determined from the vagal electroneurogram information; and
wherein in response to the processor circuit detecting the seizure event or determining that a seizure event is imminent based on the determined likelihood, the processor circuit is configured to control the signal generator circuit to generate a VNS therapy signal.

25. The seizure management system of claim 24, wherein the VNS system comprises a first electrode configured for implantation at or near a first neural target in the patient, wherein the first electrode is configured to provide the VNS therapy signal from the signal generator circuit to the first neural target; wherein the VNS system comprises a second electrode configured for implantation at or near a second neural target in the patient, wherein the second electrode is configured to receive electrical activity information from a vagus nerve of the patient, and wherein the sensor circuit is coupled to the second electrode.

26-28. (canceled)

29. The seizure management system of claim 24, wherein the machine learning-based model is configured to identify a particular pattern in the historical VENG information that correlates with prior patient seizures, and wherein the processor circuit is configured to monitor subsequent VENG information from the patient for the same particular pattern, and wherein in response to recognizing the same particular pattern in the subsequent VENG information, the processor circuit is configured to provide an alert to the patient or a caregiver.

30. (canceled)

31. The seizure management system of claim 29, wherein the sensor circuit is configured to receive information about a patient heart rate; and
wherein the machine learning-based model is configured to identify the particular pattern that correlates with prior patient seizures based on the information about the patient heart rate and the historical VENG information.

32-39. (canceled)

40. The seizure management system of claim 24, wherein the multiple physiologic parameters of the patient further comprise one or more of heart rate information or heart rate variability information or heart palpitation information or respiratory information about the patient, and wherein the information about the multiple physiologic parameters is received using the sensor circuit of the implantable VNS system.

41-43. (canceled)

44. The seizure management system of claim 24, wherein the multiple physiologic parameters of the patient further comprise patient-reported information about gastrointestinal, genitourinary, and/or cutaneous sensations experienced by the patient.

45. The seizure management system of claim 24, wherein the machine learning-based model is trained using vagal electroneurogram information from multiple patients.

46-48. (canceled)
